(19) United States
(12) Patent Application Publication
(10) Pub. No.: US 2025/0235348 A1
(43) Pub. Date: Jul. 24, 2025

(54) INSULATED POUCH WITH ICE PACK POCKETS

(71) Applicant: Rochelle BRAY, Newtown, PA (US)

(72) Inventor: Rochelle BRAY, Newtown, PA (US)

(21) Appl. No.: 18/417,061

(22) Filed: Jan. 19, 2024

(51) Int. Cl. A61F 7/10

(52) U.S. Cl. CPC A61F 7/103; A61F 2007/105; A61F 2007/108

(57) ABSTRACT

An insulated pouch with ice pack pockets is shown and described. The insulated pouch with ice pack pockets includes an insulated pouch having at least one layer. A first exterior layer, a second layer and a third interior layer. The second layer is comprised of at least an insulation. The insulated pouch is configured to have an interior with a volume capable of fitting a human body therein. An opening located at least at one end of the insulated pouch. A plurality of pockets attached to the interior of the insulated pouch. An ice pack located within each of the plurality of pockets. In one instance the ice packs are removable from each pocket.

### BACKGROUND OF THE INVENTION

The present invention relates to icing one's body in a manner similar to an ice plunge or ice bath. More particularly, the present invention provides an insulated pouch with ice pack pockets such that an individual may experience all of the benefits of an ice bath but without the need for smaller ice cubes and water.

Athletes have long been using ice or ice water as a means of maintaining and aiding their body in recovery after a workout or big game. In many instances athletes submerge themselves in an ice bath or use a machine to apply ice water to sore, swollen or injured areas. In recent years this use of ice or super cold water has been used by the general public for all sorts of reasons. In general cooling the body is a good way to help reduce swelling and heal damaged or sore areas.

There are several different ways ice or ice water has been applied to a body in the past. In one instance a large tub may be filled with ice and water, in some instances a tub with a jet is used to circulate water, sometimes referred to as a whirlpool. Using a tub and ice can be difficult, it take a large amount of ice to cool a tub of water to a degree which properly cools the body. Further, one must submerge themselves in the water in order to experience the effects. This can make an ice bath difficult or impossible for someone who travels. Further, whirlpools or other such items are large, expensive, and incapable of easy transport, or transport at all.

There have also been several different machines created to apply ice water to a limited area such as a shoulder or an angle. These devices are meant for a smaller area and not the entire body. These devices are also costly. In addition these devices still require ice cubes and water in order to function. This does not meeting the needs of someone looking to ice their entire body.

The most basic form of applying ice to a body is via ice chips or cubes and some sort of bag or container. These too have drawbacks. When using a bag or container of ice the container always tends to leak as the ice melts. Further, the ice bag seldom stays in the desired location. Both of these issues make icing a specific location difficult or annoying. In addition, it would take hundreds of pounds of ice to properly ice one's entire body using ice and bags. While functional for a small area, using ice bags is not a good way to cool one's entire body.

Consequently, there is a need for an improvement in the art of applying ice or another type of cold environment to an individual's body. The present invention substantially diverges in design elements from the known art while at the same time solves a problem many people face when wishing to ice their body without ice cubes or water. In this regard the present invention substantially fulfills these needs.

### SUMMARY OF THE INVENTION

The present invention provides an insulated pouch with ice pack pockets wherein the same can be utilized for providing convenience for the user when looking for the benefits of an ice plunge without the water. The insulated pouch with ice pack pockets includes an insulated pouch comprised of at least one layer. The insulated pouch is configured to have an interior with a volume capable of fitting a human body therein. The insulated pouch has an opening located at least at one end. A plurality of pockets attached to the interior of the insulated pouch. An ice pack located within each of the plurality of pockets.

Another object of the insulated pouch with ice pack pockets is to have three layers. A first exterior layer, a second layer and a third interior layer. The second layer is comprised of at least an insulation.

Another object of the insulated pouch with ice pack pockets is to have the ice packs be removably placed within each pocket.

Another object of the insulated pouch with ice pack pockets is to have two sets of pockets located on a top side of the interior and one located on a bottom side of the interior.

Another object of the insulated pouch with ice pack pockets is to have six pockets in each of the first set of pockets and the second set of pockets.

Another object of the insulated pouch with ice pack pockets is to have each of the ice packs are flexible.

Another object of the insulated pouch with ice pack pockets is to have a pad located above the opening to the insulated pouch,

Another object of the insulated pouch with ice pack pockets is to have the opening extend down one side of the insulated pouch.

Another object of the insulated pouch with ice pack pockets is to have a closure device located along the opening.

Another object of the insulated pouch with ice pack pockets is to have an additional pocket located within the pad.

Another object of the insulated pouch with ice pack pockets is to have a neck and head support device configured to fit within the additional pocket.

The Another object of the insulated pouch with ice pack pockets is to have a side pocket.

Another object of the insulated pouch with ice pack pockets is to have a pad configured to fit within the side pocket.

Another object of the insulated pouch with ice pack pockets is to have the pad is configured to be contoured to a body.

Another object of the insulated pouch with ice pack pockets is to have is to have insulated pouch only include three layers, a first exterior layer, a second layer and a third interior layer. The second layer is comprised of at least an insulation. The insulated pouch is configured to have an interior with a volume capable of fitting a human body therein. The insulated pouch has an opening located at least at one end, wherein the opening extends down one side of the insulated pouch. A plurality of pockets attached to the interior of the insulated pouch. An ice pack located within each of the plurality of pockets.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a top-down view of an embodiment of the insulated pouch with ice pack pockets.

FIG. 1B shows a top-down view of an embodiment of the insulated pouch with ice pack pockets.

FIG. 2 shows a top-down view of the insulated pouch with ice pack pockets in an open configuration.

FIG. 3 shows a perspective view of an embodiment of the insulated pouch with ice pack pockets.

FIG. 4 shows a perspective in use view of an embodiment of the insulated pouch with ice pack pockets.

### LIST OF REFERENCE NUMERALS

With regard to the reference numerals used, the following numbering is used throughout the drawings.

| | |
|---|---|
| 101 | Pouch/Insulated Pouch |
| 101a | Shoulder Area |
| 101b | Foot Area |
| 102 | First Layer |
| 103 | Second Layer |
| 104 | Third Layer |
| 105 | Opening |
| 106 | Pad |
| 107 | Drawstring |
| 108 | Sealing Device |
| 109 | Single Layer |
| 201 | Pockets |
| 201a | Open Edge |
| 202 | Ice Pack |
| 301 | Additional Pocket |
| 301a | Closure Device |
| 302 | Head Support |
| 303 | Side Pocket |
| 303a | Closure Device |
| 304 | Pad |

### DETAILED DESCRIPTION OF THE INVENTION

Reference is made herein to the attached drawings. Like reference numerals are used throughout the drawings to depict like or similar elements of the insulated pouch with ice pack pockets. For the purposes of presenting a brief and clear description of the present invention, a preferred embodiment will be discussed as used for the insulated pouch with ice pack pockets. The figures are intended for representative purposes only and should not be considered to be limiting in any respect.

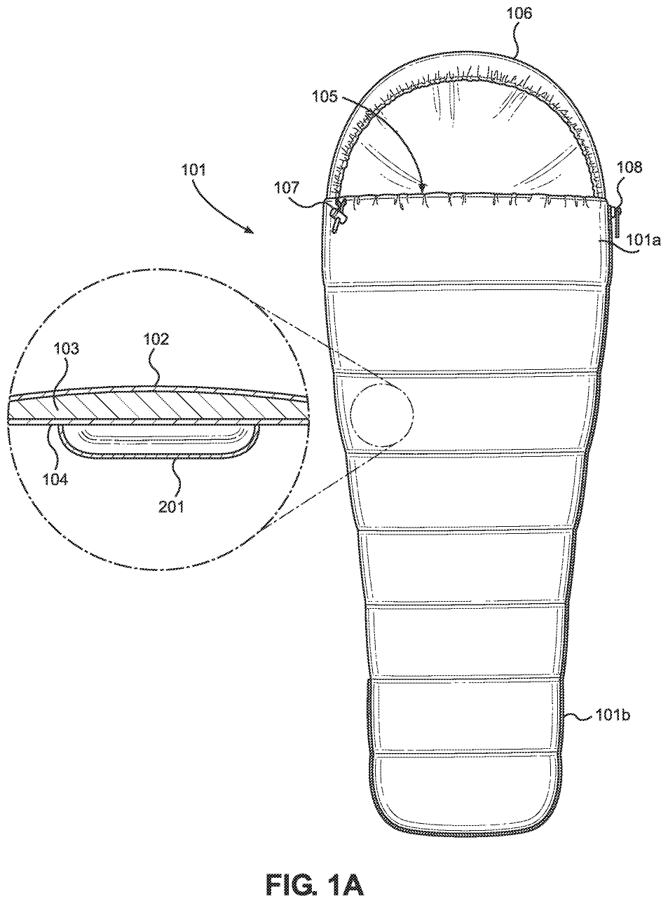

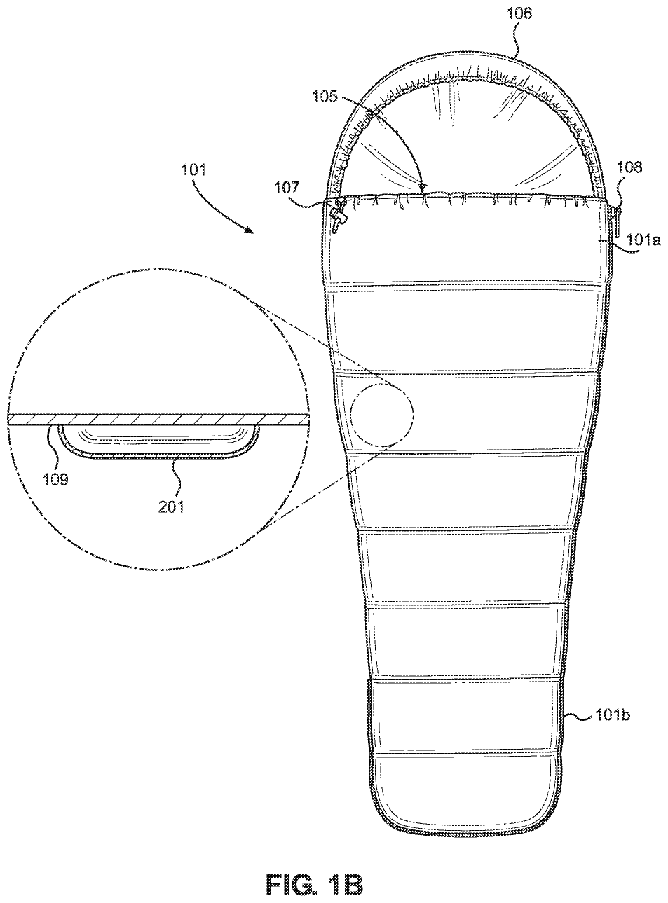

Referring now to FIG. 1A, there is shown a top-down view of an embodiment of the insulated pouch with ice pack pockets. In different embodiments the insulated pouch 101 may take different shapes. However, in general the insulated pouch 101 is of a size that will fit a human body therein from head to toe. In the shown embodiment the insulated pouch 101 is of a shape similar to a mummy sleeping bag. The insulated pouch 101 is wider in the shoulder area 101a and narrower in the foot area 101b. This shape helps to reduce size of the insulated pouch 101 for easier travel and storage. This shape also helps to reduce the area needed to cooled within the insulated pouch 101.

In the shown embodiment of FIG. 1A the insulated pouch 101 is made from three layers and a plurality of pockets. In FIG. 1A there is a call out showing the first layer 102. The first layer 102 may be constructed from various materials. In one embodiment the first layer 102 is made from ripstop nylon. This material will hold up to heavy use and at the same time maintain a light weight design.

In the callout of FIG. 1A there is also shown a second layer 103. In one embodiment the second layer 103 is an insulation layer. In one embodiment the insulation may be a synthetic insulation. The insulation will help to maintain a lower temperature within the insulated pouch 101. In another embodiment the insulation is a natural material such as down. In different embodiments the insulation will be of varying thickness. However, in general the second layer 103 includes enough insulation to maintain a temperature which is lower than standard body temperature, 98.2 degrees Fahrenheit, for at least 20 minutes.

The insulated pouch 101 includes a third layer 104. The third layer 104 will be the interior layer. In one embodiment the third layer 104 is also made from a ripstop nylon for the same reasons as provided above. In another embodiment the third layer 104 is made from a material which could be considered comfortable to the touch. This material could be for example, cotton, wool, satin, or a similar synthetic material.

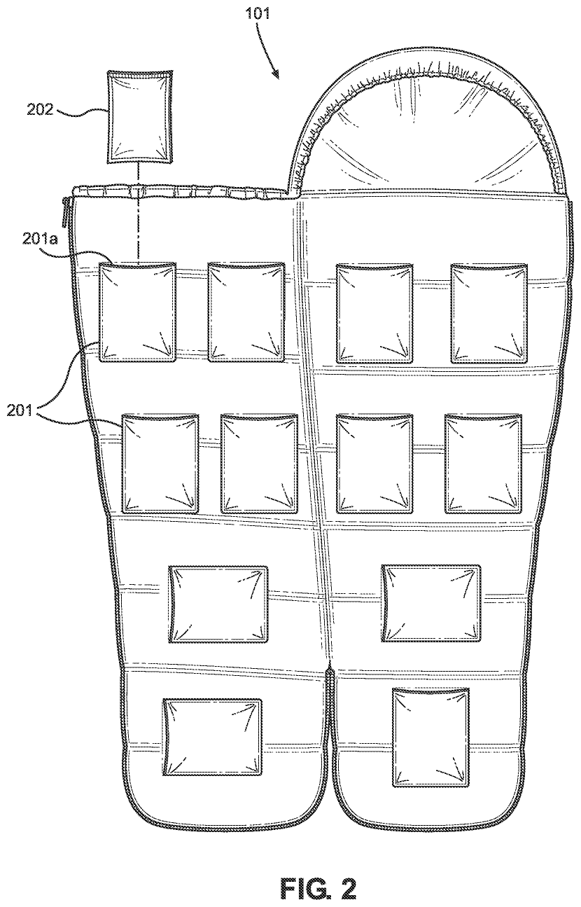

This embodiment also shows a pocket 201 which is attached to the third layer 104 on what will be the interior of the insulated pouch 101. The pocket 201 will be further detailed in the description of FIG. 2.

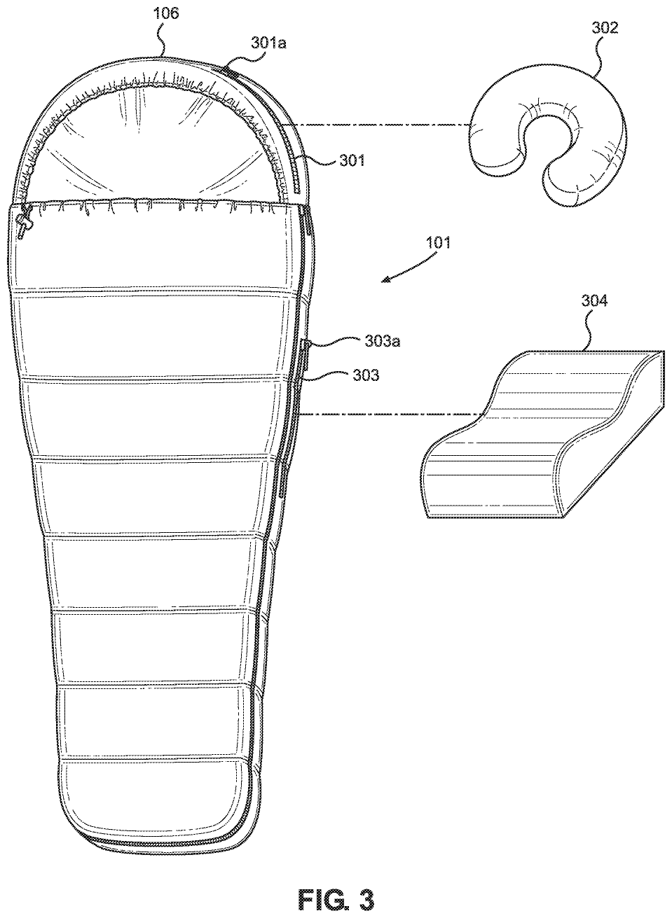

In the shown embodiment the insulated pouch further includes an opening 105. The opening 105 will be in a location where an individual would place their head. In one embodiment the a pad 106 extends past the opening 105. In one embodiment the pad 106 includes the same layers as the rest of the insulated pouch 101. In other embodiments additional layers or pouches may be included in the pad 106. These are discussed in the description of FIG. 3. In the shown embodiment a drawstring 107 is secured about the perimeter of the pad 106. The drawstring 107 may be used to close the pad 106 about one's head. This will in part seal the opening 105 closed.

In one embodiment the insulated pouch 101 is fully sealed such that the only opening is located at the top part thereof. In another embodiment the insulated pouch 101 my open into roughly half. This will allow for easier access to the inside of the insulated pouch 101. In one embodiment the insulated pouch includes a sealing device 108. In one instance the sealing device 108 may be a zipper. In another the sealing device 108 may be Velcro.

In one embodiment as shown in FIG. 1B, the insulated pouch with ice pack pockets is not necessarily insulated in the common sense. In this embodiment the pouch 101 still includes the similar features to that of the embodiment of FIG. 1A. For example, the pouch 101 includes an opening 105. The opening 105 still may include a pad 106 which extends upward therefrom. In addition, the pouch 101 may still include a drawstring 107 in order to help seal the opening 105 about a user. In one embodiment the pouch 101 has a closure device 108 which will extend at least partially down one side of the pouch 101.

In one embodiment as discussed above and shown in FIG. 1A the pouch 101 is made from several layers and includes insulation in the common sense of the term. In the embodiment shown in FIG. 1B, however, the pouch may be made having at least one layer 109. The plurality of pockets 201 are then connected to this single layer 109. In one embodiment the single layer is made from a nylon material. In another embodiment the single layer 109 is fleece. This layer may still have insulative properties in the sense that the internal temperature of the pouch will be kept lower than the surrounding air.

Referring now to FIG. 2, there is shown a top-down view of the insulated pouch with ice pack pockets in an open configuration. In one embodiment the insulated pouch 101 may be opened by folding one half of the pouch away from another. In another embodiment the insulated pouch 101 may be turned inside out. The shown embodiment is one where the insulated pouch 101 may be opened for improved access to the interior. However, the present description applies to any form of the insulated pouch 101.

The interior of the insulated pouch 101 includes a plurality of pockets 201. each of the plurality of pockets is secured to the interior of the insulated pouch 101. In one embodiment the plurality of pockets 201 are secured on three sides having one open edge 201a. In one embodiment the open edge 201a has an elastic quality. Each of the plurality of pockets 201 is configured to accept an ice pack 202 therein. The elastic quality located at one edge 201a will better secure the ice pack 202 therein.

In one embodiment there may be six pockets 201 located on each side of the interior of the of the insulated pouch 101. In other embodiments different numbers of pockets 201 may be used. The number of pockets 201 is directly related to the size of the ice packs 202. For example, in the shown embodiments the ice packs 202 may be of a size which is 12 inches by 18 inches. This means that the ice packs 202 would cover an area of approximately 24 inches by 36 inches around the shoulder and torso area and approximately 18 inches by 24 inches around the leg and foot area.

If the pockets 201 and the ice packs 202 were to be 6 inches by 6 inches the number of pockets would double and triple respectively to cover the same or similar surface area. In order to cover most of the average human body the ice packs 202 would want to cover an area which is approximately 2 to 3 feet wide and 2 to 3 feet long around the shoulder and torso area. Similarly, the ice packs would want to cover approximately 18 inches to 24 inches wide and approximately 2 to 3 feet long in the leg and foot area. These dimensions are only examples and one of ordinary skill in the art will recognize that different body, shapes, sizes, and types may benefit from different sizes, locations, and coverage areas of ice packs 202.

In different embodiments different numbers of ice pack pockets 201 may be used. The number of ice pack pockets 201 will be dependent upon the surface area the user desired to be covered by ice packs 202 and the size of the ice packs 202. If the ice packs 202 are smaller for example, more ice pack pockets 201 may be required to cover a surface area which would be the same if the ice packs 202 were larger. Further, more ice pack pockets 201 may be required if one needed to cover a larger surface area and the ice pack 202 size remained the same. This disclosure is not meant to limit the size or number of ice packs 202 used or ice pack pockets 201 used, only to be an example of configurations which could be used. One of ordinary skill in the art will be able to determine the number of ice packs 202 and ice pack pockets 201 to use.

In different embodiments different types of ice packs 202 may be used. However, the ice packs 202 need to be of a construction where they will not burst or tear under the weight of a human body. In one embodiment the ice packs 202 are made from a solid material. In another embodiment the ice packs 202 are made from a flexible material. When the ice packs are made from a flexible material and include a substance that reaches a temperature of ice but remains flexible the ice packs 202 will be able to conform to a body's shape and contour.

Referring now to FIG. 3, there is shown a perspective view of an embodiment of the insulated pouch with ice pack pockets. In some embodiments the insulated pouch with ice pack pockets includes different pads or supports to better allow for an individual to be in a comfortable position while using the device. In some embodiments the additional items may also allow for the ice packs to be pressed to the body.

In one embodiment the insulated pouch 101 includes an additional pocket 301 located in the pad 106. In one embodiment the additional pocket 301 includes a closure device 301a. In one embodiment the closure device 301a is a zipper. In another embodiment the closure device 301a is a hook and loop fastener.

In this embodiment the additional pocket 301 is configured to receive a neck and head support 302. In one embodiment the neck and head support 302 is a pillow. In another embodiment the neck and head support 302 is a foam pad which will contour with and support the head and neck of a user.

In one embodiment the insulated pouch 101 includes a side pocket 303 located along the side of the insulated pouch 101. The side pocket 303 is located such that an item may be placed between the layers of the insulated pouch 101 on a bottom side thereof. In one embodiment the side pocket 303 includes a closure device 303a. In one embodiment the closure device 303a is a zipper. In another embodiment the closure device 303a is a hook and loop fastener.

In one embodiment the side pocket 303 is configured to accept a pad 304 therein. In one embodiment the pad simply adds a cushion to the insulated pouch 101. In another embodiment the pad 304 is configured to support a user's back. In one embodiment the pad 304 is configured to support the natural curve of one's back and hips. In this embodiment the ice packs will be given extra support to better be placed against the body.

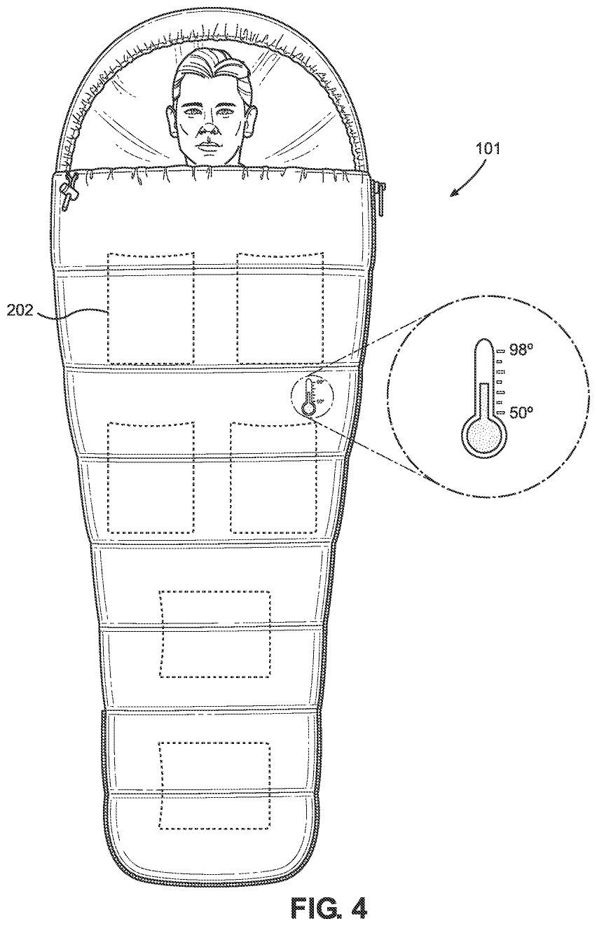

Referring now to FIG. 4, there is shown a perspective in use view of an embodiment of the insulated pouch with ice pack pockets. In use an individual places ice packs 202 into each of the pockets on the interior of the insulated pouch 101. Each of the ice packs 202 being in a frozen condition will lower the temperature of the interior of the insulated pouch 101.

An individual then places themselves into the insulated pouch 101. Once inside the ice packs will press against, or be pressed against, the body. This will cool the entire body. After a length of time, as determined by the user, the user exits the insulated chamber 101. In this manner a user can experience all of the effects and results of an ice plunge without the need for ice or water.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An insulated pouch for physical therapy comprising:
a first sheet positioned on a second sheet at is positioned on a third interior sheet with each sheet having opposing short sides and opposing long sides and the second sheet being comprised of at least an insulation material;
an interior formed by securing a long side of the first sheet, second sheet and third sheet to the opposing long side of the first sheet, second sheet and third sheet to create a volume capable of fitting a human body therein;
an opening on a short side of the first sheet, second sheet and third sheet providing access into the interior volume with the opening being sized to accommodate a human head that is exposed to the outside through the opening when a person is inside the interior volume with the opposing side of the interior volume being closed;
a plurality of pockets attached to the interior of the insulated pouch with the pockets being arranged and aligned horizontally and vertically on the third interior sheet;
an ice pack located within each of the plurality of pockets.

2. The insulated pouch of claim 1, wherein the ice packs are removably placed within each pocket.

3. The insulated pouch of claim 1, wherein there are two sets of pockets located on a top side of the interior and one located on a bottom side of the interior.

4. The insulated pouch of claim 3, wherein there are six pockets in each of the first set of pockets and the second set of pockets.

5. The insulated pouch of claim 1, wherein each of the ice packs are flexible.

6. The insulated pouch of claim 1, including a pad located above the opening to the volume.

7. The insulated pouch of claim 1, wherein the opening extends down one side of the insulated pouch.

8. The insulated pouch of claim 7, including a closure device located along the opening.

9. The insulated pouch of claim 6, including an additional pocket located within the pad.

10. The insulated pouch of claim 9, including a neck and head support device configured to fit within the additional pocket.

11. The insulated pouch of claim 1, including a side pocket.

12. The insulated pouch of claim 11, including a pad configured to fit within the side pocket.

13. The insulated pouch of claim 12, wherein the pad contours to a body.

14. An insulated pouch for physical therapy consisting of:
at least one layer of material;
an interior with a volume capable of fitting a human body therein;
an opening located at least at one end that is sized to accommodate a human head that is exposed to the outside through the opening when a person is inside the interior volume, wherein the opening extends down one side of the insulated pouch;
a plurality of pockets attached to the interior of the volume;
an ice pack located within each of the plurality of pockets.

15. An insulated pouch for physical therapy, the insulated pouch comprising:
at least one layer of material;
the insulated pouch is configured to have an interior with a volume capable of fitting a human body therein;
an opening located at least at one end with the opening being sized to accommodate a human that is exposed to the outside through the opening head when a person is inside the interior volume;
a plurality of pockets attached to the interior of the insulated pouch;
an ice pack located within each of the plurality of pockets.

16. The insulated pouch of claim 15, wherein the ice packs are removably placed within each pocket.

17. The insulated pouch of claim 15, wherein each of the ice packs are flexible.

18. The insulated pouch of claim 15, further comprising a pad located above the opening to the insulated pouch.

19. The insulated pouch of claim 15, wherein the opening extends down one side of the insulated pouch.
